(19) United States
(12) Patent Application Publication
(10) Pub. No.: US 2013/0190404 A1
(43) Pub. Date: Jul. 25, 2013

(54) CRYSTALS OF CARBOPROST TROMETHAMINE AND THE PREPARATION METHOD AS WELL AS THE USES THEREOF

(71) Applicants: Ming Li, Shanghai (CN); Zhijun Tang, Shanghai (CN); Xiaoming Ji, Shanghai (CN); Xiaoliang Gao, Shanghai (CN)

(72) Inventors: Ming Li, Shanghai (CN); Zhijun Tang, Shanghai (CN); Xiaoming Ji, Shanghai (CN); Xiaoliang Gao, Shanghai (CN)

(73) Assignee: SHANGHAI TECHWELL BIOPHARMACEUTICAL CO., LTD., Shanghai (CN)

(21) Appl. No.: 13/811,200

(22) PCT Filed: Jul. 20, 2011
(86) PCT No.: PCT/CN11/77371

§ 371 Date: Apr. 9, 2013

(30) Foreign Application Priority Data
Jul. 21, 2010 (CN) 201010232026.0

(51) Int. Cl. C07C 59/46; C07C 215/10

(52) U.S. Cl. CPC C07C 59/46; C07C 215/10
USPC 514/573; 562/503

(57) ABSTRACT

Disclosed is a crystal of carboprost tromethamine as represented by Formula (I). The crystal has characteristic peaks in the X-ray diffraction pattern at the following 2θ angles: 6.6±0.2°, 9.9±0.2°, 18.5±0.2° and 20.1±0.2°. Furthermore, also disclosed are preparation method and the use of the crystal.

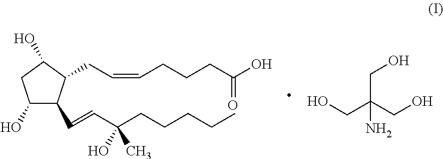 [SMILES: CCCCC[C@](C)(O)/C=C/[C@@H]1[C@@H](C/C=C\CCCC(=O)O)[C@@H](O)C[C@H]1O ; NC(CO)(CO)CO]

### TECHNICAL FIELD

The present invention relates to crystals of a compound. Particularly, the invention relates to the crystals of carboprost tromethamine and the preparation methods as well as the uses thereof.

### BACKGROUND ART

Carboprost tromethamine, i.e., 2-amino-2-(hydroxymethyl)propan-1,3-diol(5Z)-7-[(1R,2R,3R,5S)-3,5-dihydroxy-2-[(1E,3S)-3-hydroxy-3-methyl-1-octene]cyclopentyl]-5-heptylic acid, belongs to prostaglandins.

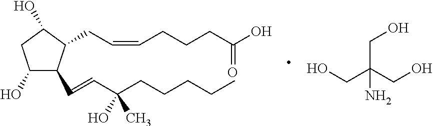 [SMILES: CCCCC[C@](C)(O)/C=C/[C@@H]1[C@@H](C/C=C\CCCC(=O)O)[C@@H](O)C[C@H]1O ; NC(CO)(CO)CO]

Carboprost tromethamine is mainly used to treat abortion during week 13-20 of pregnancy period and refractory postpartum uterine bleeding. For the postpartum bleeding caused by uterus contraction retard to which conventional treatment is ineffective, carboprost tromethamine has significant effect.

The purity and uniformity requested by the drug administration can not be readily achieved by using amorphous compounds during the preparation process of drugs, and amorphous compounds are inferior to the crystalline compounds in stability, therefore, it is urgent to develop the crystal of carboprost tromethamine suitable for the use in the preparation of drugs.

### SUMMARY OF THE INVENTION

The purpose of the present invention is to provide the crystals of carboprost tromethamine.

Another purpose of the present invention is to provide the preparation methods for the above crystals.

The further purpose of the present invention is to provide the use of the above crystals.

In the first aspect of the present invention, a crystal of carboprost tromethamine of formula I is provided, wherein said crystal has characteristic peaks at the following 2θ angles in the X-ray diffraction pattern: 6.6±0.2°, 9.9±0.2°, 18.5±0.2° and 20.1±0.2°,

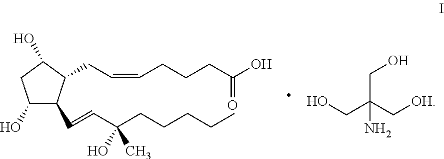 [SMILES: CCCCC[C@](C)(O)/C=C/[C@@H]1[C@@H](C/C=C\CCCC(=O)O)[C@@H](O)C[C@H]1O ; NC(CO)(CO)CO]

In another preferred embodiment, said crystal has other characteristic peaks at the following 2θ angles in the X-ray diffraction pattern: 19.3±0.2°, 19.5±0.2°, 19.9±0.2° and 21.6±0.2°.

In another preferred embodiment, said crystal has other characteristic peaks at the following 2θ angles in the X-ray diffraction pattern: 13.3±0.2°, 15.8±0.2°, 16.7±0.2°, 17.7±0.2°, 18.1±0.2°, 20.8±0.2°, 21.1±0.2°, 26.9±0.2°, 27.6±0.2°, 33.8±0.2° and 40.8±0.2°.

The maximum peak is at 103.97±5° C. in the differential scanning calorimetry (DSC) for said crystal of carboprost tromethamine.

Infrared Spectrum of said crystal of carboprost tromethamine is shown in FIG. 3.

In the second aspect of the present invention, a method for preparing the crystal of carboprost tromethamine according to the invention is provided, said method including the following steps:

a. dissolving the carboprost of formula II in solvent (i) to obtain solution 1;

b. adding the aqueous trometamol of formula III into solution 1 dropwise to obtain said crystal of carboprost tromethamine according to the invention;

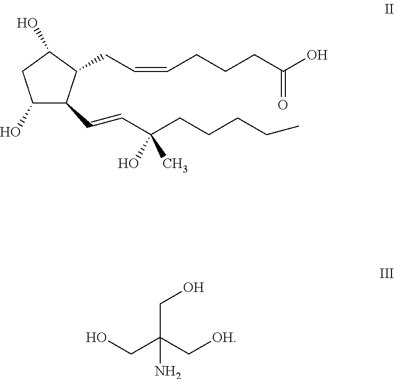 [SMILES: CCCCC[C@](C)(O)/C=C/[C@@H]1[C@@H](C/C=C\CCCC(=O)O)[C@@H](O)C[C@H]1O ; NC(CO)(CO)CO]

In step a of said method, said solvent (i) is selected from at least one of the following group: acetonitrile, acetone, ethyl ether, C1-4 straight or branched chain alcohol; the temperature for dissolving the carboprost in step a is 0° C.-100° C.; and the amount of solvent (i) in step a is 1000:1-1:1 (ml solvent/g carboprost).

In step b of said method, when adding the aqueous trometamol into solution 1 dropwise, the temperature is 20° C.-100° C.; the molar ratio of trometamol added in step b and carboprost is 0.8:1-1.2:1; and the amount of water added in step b is 0.01:1-10:1 (ml water/g carboprost).

In another preferred embodiment, in step b, after the aqueous trometamol is added dropwise into solution 1, the mixture resulted is refluxed for 10 mins with mixing, and cooled slowly to precipitate the crystal of carboprost tromethamine according to the invention.

In another preferred embodiment, said method includes the following steps:

a. dissolving the carboprost of formula II in solvent (i) to obtain solution 1;

b. adding the aqueous trometamol of formula III into solution 1 dropwise to obtain the crystal of carboprost tromethamine;

c. dissolving the resulted crystal in the water to obtain solution 2; and

d. adding acetone into solution 2 dropwise to obtain said crystal of carboprost tromethamine according to the invention.

In another preferred embodiment, steps c and d are conducted under the temperature of −30° C. to 30° C.

In another preferred embodiment, the amount of water added in step c is 0.1:1 to 10:1 (ml water/g carboprost tromethamine).

In another preferred embodiment, the amount of acetone added in step d is 1:1 to 100:1 (ml acetone/g carboprost tromethamine).

In the third aspect of the present invention, a pharmaceutical composition is provided, wherein said composition comprises said crystal of carboprost tromethamine according to the invention and pharmaceutically acceptable carriers.

In the fourth aspect of the present invention, the use of said crystal of carboprost tromethamine according to the invention is provided, wherein said crystal is used to prepare the medicaments for preventing and treating postpartum bleeding and abortion during the first and second trimester pregnancy as well as artificial abortion.

Based on the above, the crystal of carboprost tromethamine suitable for the preparation of medicaments is provided by the present invention.

### BRIEF DESCRIPTION OF THE DRAWINGS

FIG. 1 is the X-ray powder diffraction pattern of the crystal of carboprost tromethamine according to the present invention, wherein the ordinate indicates the X-ray intensities in cps, and the abscissa indicates the diffraction angle in 2θ.

FIG. 2 is the differential scanning calorimetry (DSC) of the crystal of carboprost tromethamine according to the present invention.

FIG. 3 is the infrared Spectrum of the crystal of carboprost tromethamine according to the present invention.

### DETAILED DESCRIPTION OF THE INVENTION

Upon research, the inventors have surprisingly discovered that the purity and uniformity requested by the drug administration can be readily achieved by using a crystal of carboprost tromethamine, and the crystal possesses surprisingly good stability, therefore, the crystal is particularly suitable for the preparation of medicaments. Thus, the present invention has been accomplished.

As used herein, the term “crystal” refers to the solid of a molecule or atom complex showing specific arrangement.

In X-ray polycrystal diffraction, i.e., X-ray powder diffraction, a series of diffraction phenomena can be produced when X-ray passing through a crystal. In the laboratories, X-ray diffraction pattern can be obtained by using X-ray diffractometer. In the pattern, different diffraction lines and the intensities thereof are determined by atomic cluster having certain structure. The diffraction patterns produced by the crystal with different structures will be different from each other, therefore, the structure of a crystal can be determined by its X-ray diffraction pattern, i.e., crystal form.

The methods for determining the X-ray diffraction pattern of a crystal are known in the art. For example, X-ray diffraction pattern can be obtained by using RIGAKU D/max 2550VB/PC X-ray powder diffractometer with the scanning rate of 2°/min. Copper irradiated target is used.

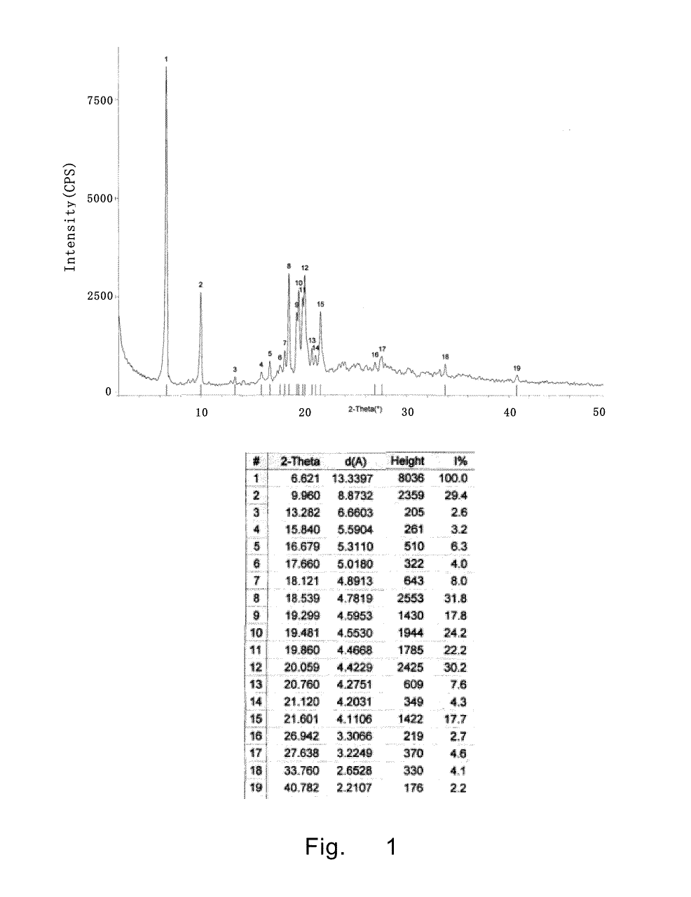

The crystal of carboprost tromethamine according to the present invention possesses the unique crystal form. And such crystal possesses specific characteristic peaks in the X-ray diffraction pattern. Particularly, the crystal of carboprost tromethamine according to the present invention possesses characteristic peaks at the following 2θ angles in the X-ray powder diffraction pattern: 6.6±0.2°, 9.9±0.2°, 13.3±0.2°, 15.8±0.2°, 16.7±0.2°, 17.7±0.2°, 18.1±0.2°, 18.5±0.2°, 19.3±0.2°, 19.5±0.2°, 19.9±0.2°, 20.1±0.2°, 20.8±0.2°, 21.1±0.2°, 21.6±0.2°, 26.9±0.2°, 27.6±0.2°, 33.8±0.2° and 40.8±0.2°. Preferably, said crystal possesses the X-ray diffraction pattern substantially identical with FIG. 1.

“Differential scanning calorimetry” (DSC) is a technology for measuring the relationship of energy difference between the tested substance and the reference and temperature during the heating process. On the DSC pattern, the location, form and number of the peak are relevant to the properties of the substance, therefore, the substance can be qualitatively identified by using DSC. Said method is used in the art to detect many parameters of a substance, such as the phase transition temperature, glass transition temperature and reaction heat.

DSC is known in the art. For example, DSC pattern of a crystal can be obtained by using DSC Q20 differential scanning calorimeter under the following conditions: warming rate of 10° C./min, from 25° C. to 300° C.

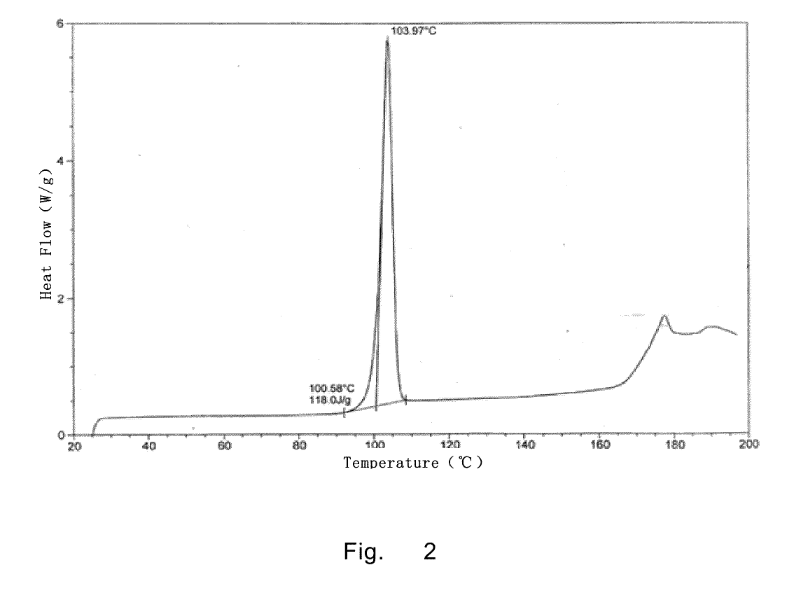

In one embodiment of the present invention, the crystal of carboprost tromethamine obtained by the method according to the present invention was determined to have the maximum peak at 103.97±5° C. by DSC. Preferably, the crystal of carboprost tromethamine according to the present invention has the DSC pattern substantially identical with FIG. 2. More preferably, the maximum peak can be found at 103.97° C. in the DSC pattern.

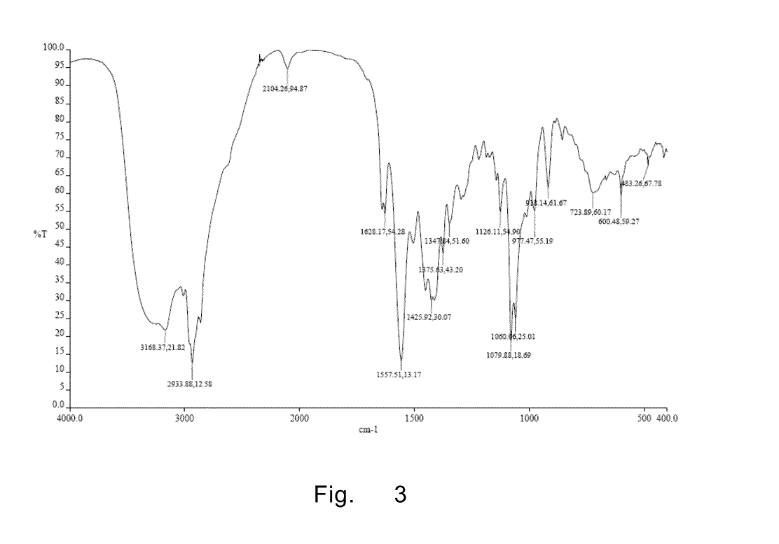

The crystal structure can also be determined by Infrared Spectrometry (IR), which is known in the art. For example, it can be determined by using PE Spectrum One B, tableting at KBr: sample=200:1, and scanning 400˜4000 cm⁻¹. The crystal of carboprost tromethamine according to the present invention has characteristic peaks at the following wave numbers: 3168.37 cm⁻¹, 2933.88 cm⁻¹, 2104.26 cm⁻¹, 1628.17 cm⁻¹, 1557.51 cm⁻¹, 1425.92 cm⁻¹, 1375.63 cm⁻¹, 1347.84 cm⁻¹, 1126.11 cm⁻¹, 1079.88 cm⁻¹, 1060.06 cm⁻¹, 977.47 cm⁻¹, 918.14 cm⁻¹, 723.89 cm⁻¹, 600.48 cm⁻¹, 483.26 cm⁻¹. Preferably, the crystal has the IR pattern substantially identical with FIG. 3.

A method for the preparation of said crystal is provided, and generally, said method includes the following steps:

a. dissolving the carboprost in solvent (i) to obtain solution 1;

b. adding the aqueous trometamol into solution 1 dropwise, followed by cooling slowly to obtain the crystal of carboprost tromethamine.

In one embodiment, solvent (i) in step a is selected from at least one of the following group: acetonitrile, acetone, ethyl ether, C1-4 straight or branched chain alcohol.

In another embodiment, the carboprost is dissolved in solvent (i) in step a at 0° C.-100° C., preferably 30° C.-60° C.

In another embodiment, the amount of solvent (i) added in step a is 1000:1-1:1 (ml solvent/g carboprost), preferably 200:1-100:1 (ml solvent/g carboprost).

In another embodiment, the aqueous trometamol is added into solution 1 dropwise in step b at 20° C.-100° C., preferably 35° C.-65° C.

In another embodiment, the molar ratio of trometamol added in step b and carboprost is 0.8:1-1.2:1, preferably, 1:1.

In another embodiment, the amount of pure water added in step b is 0.01:1-10:1 (ml water/g carboprost); preferably, 0.5:1-1:1 (ml water/g carboprost).

In another embodiment, the amount of pure water in the aqueous trometamol in step b is 0.5:1-10:1 (ml water/g trometamol); preferably, 1.0:1-5:1 (ml water/g trometamol).

Preferably, in step b, the aqueous trometamol is added dropwise into solution 1, the resulted mixture is heated to reflux, and then cooled slowly, thereby obtaining the crystal of carboprost tromethamine.

In another example according to the present invention, said method further includes the following steps after steps a and b:

c. dissolving the crystal prepared in steps a and b to obtain solution 2; and

d. adding acetone into solution 2 dropwise to obtain the crystal of carboprost tromethamine according to the present invention.

In one embodiment, steps c and d are conducted at −30° C.-30° C., preferably 0° C.-15° C.

In another embodiment, the amount of water added in step c is 0.1:1-10:1 (ml water/g carboprost tromethamine); preferably, 0.5:1-0.7:1 (ml water/g carboprost tromethamine).

In another embodiment, the amount of acetone added in step d is 1:1-100:1 (ml acetone/g carboprost tromethamine); preferably, 20:1-25:1 (ml acetone/g carboprost tromethamine).

The present invention also relates to pharmaceutical compositions or pharmaceutical formulations comprising crystalline carboprost tromethamine. Said pharmaceutical compositions or pharmaceutical formulations can be formulated into various forms suitable for oral administration or injection to treat abortion during week 13-20 of pregnancy period and refractory postpartum uterine bleeding. The preferred dosage form for pharmaceutical formulation is the injection. In addition to carboprost tromethamine, the pharmaceutical formulation according to the present invention comprises pharmaceutically acceptable carriers, excipients and/or diluents. Representative examples include (but not limited to): filler(s), such as microcrystalline cellulose, lactose, sucrose, starch, modified starch, mannose, glucan, calcium carbonate, phosphate, sulfate; binder(s), such as microcrystalline cellulose, lactose, sucrose, starch, modified starch, polyether polyols, glucan, hydroxymethyl cellulose, methyl cellulose, gelatin, polyvinylpyrrolidone, magnesium aluminum silicate; disintegrant(s), such as crosslinked polyvinylpyrrolidone, crossed linked carboxymethyl starch, starch, etc. Additionally, the formulation of the pharmaceutical composition may also comprise surfactants and colorants, if desired.

All the features mentioned above or in the examples below of the invention can be optionally combined. All features disclosed in this specification may be used in any combination. Any alternative feature serving the same, equivalent, or similar purpose may replace each feature disclosed in this specification. Therefore, unless otherwise specified, the features as disclosed are only general examples of equivalent or similar features.

The advantages of the invention mainly include:

1. The crystal of carboprost tromethamine was obtained for the first time by using the specific crystallization method of the present invention;

2. The crystal obtained by the method of the present invention possesses relatively good crystal form, higher purity as well as better chemical and physical stability.

The invention will be further illustrated with reference to the following specific examples. It is to be understood that these examples are only intended to illustrate the invention, but not to limit the scope of the invention. For the experimental methods in the following examples without particular conditions, they are performed under routine conditions or as instructed by the manufacturer. Unless otherwise specified, all percentages, ratios, proportions or parts are by weight.

The unit of the weight/volume percentages in the invention is well known to the skilled in the art, for example, the weight of a solute in a 100 mL solution.

Unless otherwise defined, all scientific and technical terms used herein have the same meaning as commonly understood by the skilled in the art. Furthermore, any process or material similar or equivalent to those described herein can be used in the process of the present invention. The preferred embodiments and materials described herein are merely provided for illustration.

In the following examples, high performance liquid chromatography (HPLC) method (European Pharmacopoeia) used for detecting the purity of the crystal of arboprost tromethamine obtained are listed as follows:

Stationary phase: silica gel, particle size 5-μm

Mobile phase: acetonitrile:aqueous Sodium Dihydrogen Phosphate (pH=2.5)=23:77 (V/V)

Flow rate: 1.0 ml/min

Column temperature: 35° C.

Detection wavelength: 200 nm

Column height: 15 cm

### Example 1

Preparation 1 of the Crystal of Carboprost Tromethamine

1 g carboprost was dissolved in 100 ml acetonitrile at 60° C. The resulted solution was warmed to 65° C., and a solution of 0.32 g trometamol in 0.5 ml pure water was added slowly dropwise. After the addition, the resulted mixture was heated to reflux for 10 mins, and then slowly cooled to the room temperature, thereby precipitating a white solid in powder. The solid was filtered, and the resulted solid was dried in a vacuum oven, thus obtaining 1.2 g of the white crystal of carboprost tromethamine. The X-ray powder diffraction pattern, the DSC pattern and the IR pattern of the crystal were identical with FIGS. 1-3, respectively.

### Example 2

Preparation 2 of the Crystal of Carboprost Tromethamine

1 g carboprost was dissolved in 100 ml acetonitrile at 60° C. The resulted solution was warmed to 65° C., and a solution of 0.32 g trometamol in 0.5 ml pure water was added slowly dropwise. After the addition, the resulted mixture was heated to reflux for 10 mins, and then slowly cooled to the room temperature, thereby precipitating 1.2 g of a white solid in powder. The resulted solid was dissolved in 0.6 ml pure water at 15° C., 24 ml acetone was added dropwise, and the resulted mixture was stirred for 0.5 h, thereby precipitating the crystal of carboprost tromethamine. The whole system was cooled to 0° C., and stirred for 2 hours. The crystal was collected by filtration, and dried in a vacuum oven, thus obtaining 1.14 g of the white crystal of carboprost tromethamine. The X-ray powder diffraction pattern, the DSC pattern and the IR pattern of the crystal were identical with FIGS. 1-3, respectively.

### Example 3

Preparation 3 of the Crystal of Carboprost Tromethamine

10 g carboprost was dissolved in 2000 ml acetone at 30° C. The resulted solution was warmed to 40° C., and a solution of 3.2 g trometamol in 10 ml pure water was added slowly dropwise. After the addition, the resulted mixture was heated to reflux for 10 mins, and then slowly cooled to the room temperature, thereby precipitating a white solid in powder. The solid was filtered, and the resulted solid was dried in a vacuum oven, thus obtaining 12.4 g of the white crystal of carboprost tromethamine. The X-ray powder diffraction pattern, the DSC pattern and the IR pattern of the crystal were identical with FIGS. 1-3, respectively.

### Example 4

Preparation 4 of the Crystal of Carboprost Tromethamine

10 g carboprost was dissolved in 2000 ml acetone at 30° C. The resulted solution was warmed to 40° C., and a solution of 3.2 g trometamol in 10 ml pure water was added slowly dropwise. After the addition, the resulted mixture was heated to reflux for 10 mins, and then slowly cooled to the room temperature, thereby precipitating 12.4 g of a white solid in powder. The resulted solid was dissolved in 6.2 ml pure water at 0° C., 248 ml acetone was added dropwise, and the resulted mixture was stirred for 0.5 h, thereby precipitating the crystal of carboprost tromethamine. The whole system was cooled to 0° C., and stirred for 2 hours. The crystal was collected by filtration, and dried in a vacuum oven, thus obtaining 11.6 g of the white crystal of carboprost tromethamine. The X-ray powder diffraction pattern, the DSC pattern and the IR pattern of the crystal were identical with FIGS. 1-3, respectively.

### Example 5

Preparation 5 of the Crystal of Carboprost Tromethamine

1 g carboprost was dissolved in 150 ml ethanol at 40° C. The resulted solution was warmed to 50° C., and a solution of 0.32 g trometamol in 0.8 ml pure water was added slowly dropwise. After the addition, the resulted mixture was heated to reflux for 10 mins, and then slowly cooled to the room temperature, thereby precipitating a white solid in powder. The solid was filtered, and the resulted solid was dried in a vacuum oven, thus obtaining 1.22 g of the white crystal of carboprost tromethamine. The X-ray powder diffraction pattern, the DSC pattern and the IR pattern of the crystal were identical with FIGS. 1-3, respectively.

### Example 6

Preparation 6 of the Crystal of Carboprost Tromethamine

1 g carboprost was dissolved in 150 ml ethanol at 40° C. The resulted solution was warmed to 50° C., and a solution of 0.32 g trometamol in 0.8 ml pure water was added slowly dropwise. After the addition, the resulted mixture was heated to reflux for 10 mins, and then slowly cooled to the room temperature, thereby precipitating 1.22 g of a white solid in powder. The resulted solid was filtered and dissolved in 0.7 ml pure water at 10° C., 248 ml acetone was added dropwise, and the resulted mixture was stirred for 0.5 h, thereby precipitating the crystal of carboprost tromethamine. The whole system was cooled to 0° C., and stirred for 2 hours. The crystal was collected by filtration, and dried in a vacuum oven, thus obtaining 1.12 g of the white crystal of arboprost tromethamine. The X-ray powder diffraction pattern, the DSC pattern and the IR pattern of the crystal were identical with FIGS. 1-3, respectively.

### Example 7

Preparation 7 of the Crystal of Carboprost Tromethamine

5 g carboprost was dissolved in 800 ml ether at 30° C. The resulted solution was warmed to 35° C., and a solution of 1.6 g trometamol in 5 ml pure water was added slowly dropwise. After the addition, the resulted mixture was heated to reflux for 10 mins, and then slowly cooled to the room temperature, thereby precipitating a white solid in powder. The solid was filtered, and the resulted solid was dried in a vacuum oven, thus obtaining 5.8 g of the white crystal of carboprost tromethamine. The X-ray powder diffraction pattern, the DSC pattern and the IR pattern of the crystal were identical with FIGS. 1-3, respectively.

### Example 8

Preparation 8 of the Crystal of Carboprost Tromethamine

5 g carboprost was dissolved in 800 ml ether at 30° C. The resulted solution was warmed to 35° C., and a solution of 1.6 g trometamol in 5 ml pure water was added slowly dropwise. After the addition, the resulted mixture was heated to reflux for 10 mins, and then slowly cooled to the room temperature, thereby precipitating 5.8 g of a white solid in powder. The resulted solid was filtered and dissolved in 3.2 ml pure water at 10° C., 128 ml acetone was added dropwise, and the resulted mixture was stirred for 0.5 h, thereby precipitating the crystal of carboprost tromethamine. The whole system was cooled to 0° C., and stirred for 2 hours. The crystal was collected by filtration, and dried in a vacuum oven, thus obtaining 5.3 g of the white crystal of arboprost tromethamine. The X-ray powder diffraction pattern, the DSC pattern and the IR pattern of the crystal were identical with FIGS. 1-3, respectively.

### Comparative-Example 1

Preparation of Amorphous Carboprost Tromethamine

1 g carboprost was dissolved in 100 ml acetonitrile at 60° C. The resulted solution was warmed to 65° C., and a solution of 0.32 g trometamol in 0.5 ml pure water was added slowly dropwise. After the addition, the resulted mixture was heated to reflux for 10 mins, and then quickly cooled by ice-water, thereby obtaining 1.2 g of viscous solid.

### Example 9

Purity and Stability Test

In the present example, the samples from the comparative-example and examples were test for purity and stability. The method for testing is described as follows:

The samples from comparative-example 1, example 2 and example 4 were obtained and kept at 25° C. for 7 days in sealed storage. And then the content of impurities in each sample was tested.

Results are shown in the following table:

Experimental conditions

| | Initial content of | Content of impurities after |
|---|---|---|
| Sample | impurities | stored at 25° C. for 7 days |
| Example 2 | 0.3% | 0.3% |
| Example 4 | 0.2% | 0.2% |
| Comparative | 0.5% | 4.0% |
| example 1 | | |

From the data shown in the table, the purity of the crystal of carboprost tromethamine is higher, and after stored for a long time, it has excellent stability.

The above examples are merely the preferred examples for the present invention, and such examples cannot be used to limit the scope of the invention. The substantial technical contents according to the present invention are broadly defined in the claims. And any entities or methods accomplished by others should be considered as the equivalents and fall within the scope as defined by the claims, if said entities or methods are the same as those defined by the claims.

1. A crystal of carboprost tromethamine according to the formula: 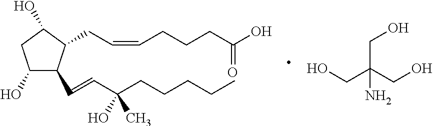
wherein, said crystal has characteristic peaks in the X-ray diffraction pattern at the following 2θ angles: 6.6±0.2°, 9.9±0.2°, 18.5±0.2° and 20.1±0.2°.

2. The crystal of carboprost tromethamine according to claim 1, wherein said crystal further has characteristic peaks in the X-ray diffraction pattern at the following 2θ angles: 19.3±0.2°, 19.5±0.2°, 19.9±0.2° and 21.6±0.2°.

3. The crystal of carboprost tromethamine according to claim 1, wherein said crystal further has characteristic peaks in the X-ray diffraction pattern at the following 2θ angles: 13.3±0.2°, 15.8±0.2°, 16.7±0.2°, 17.7±0.2°, 18.1±0.2°, 20.8±0.2°, 21.1±0.2°, 26.9±0.2°, 27.6±0.2°, 33.8±0.2° and 40.8±0.2°.

4. The crystal of carboprost tromethamine according to claim 1, wherein the maximum peak is at 103.97±5° C. in the differential scanning calorimetry (DSC).

5. The crystal of carboprost tromethamine according to claim 1, wherein the infrared Spectrum of said crystal is shown in FIG. 3.

6. A method for preparing the crystal of carboprost tromethamine of claim 1, said method comprising:
a) dissolving carboprost in a solvent to form a carboprost solution;
b) adding aqueous trometamol into said carboprost solution dropwise to obtain a crystal of carboprost tromethamine according to the formula:

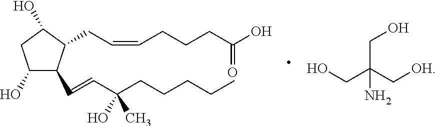 [SMILES: CCCCC[C@](C)(O)/C=C/[C@@H]1[C@@H](C/C=C\CCCC(=O)O)[C@@H](O)C[C@H]1O ; NC(CO)(CO)CO]

7. The method according to claim 6, wherein said solvent is selected from the group consisting of acetonitrile, acetone, ethyl ether, and a C1-4 straight or branched chain alcohol.

8. The method according to claim 6, wherein the temperature for dissolving the carboprost in step a is 0° C.-100° C.

9. The method according to claim 6, wherein the amount of said solvent in step (a) ranges from 1000:1 to 1:1 (ml solvent/g carboprost).

10. The method according to claim 6, wherein when adding the aqueous trometamol into said solution dropwise in step (b), the temperature is 20° C.-100° C.

11. The method according to claim 6, wherein, the molar ratio of trometamol added in step (b) to carboprost ranges from 0.8:1 to 1.2:1.

12. The method according to claim 6, wherein the amount of water added in step b ranges from 0.01:1 to 10:1 (ml water/g carboprost).

13. The method according to claim 6, wherein, in step b, after the aqueous trometamol is added dropwise into solution 1, the resulting mixture is refluxed for 10 minutes with mixing, and cooled slowly to precipitate the crystal of carboprost tromethamine.

14. The method according to claim 6, wherein said method further comprises:
c) dissolving the resulting crystal in water to obtain a second solution; and
d) adding acetone into said second solution dropwise to obtain a crystal of carboprost tromethamine.

15. The method according to claim 14, wherein steps c and d are conducted at a temperature ranging from −30° C. to 30° C.

16. The method according to claim 14, wherein the amount of water added in step (c) ranges from 0.1:1 to 10:1 (ml water/g carboprost tromethamine).

17. The method according to claim 14, wherein the amount of acetone added in step (d) ranges from 1:1 to 100:1 (ml acetone/g carboprost tromethamine).

18. A pharmaceutical composition, wherein said composition comprises the crystal of carboprost tromethamine of claim 1 and a pharmaceutically acceptable carrier.

19. A method of treating postpartum bleeding or inducing an abortion in a subject said method comprising administering a composition comprising or formulated using a crystal of carboprost according to claim 1.
